(19) United States
(12) Patent Application Publication
(10) Pub. No.: US 2018/0092930 A1
(43) Pub. Date: Apr. 5, 2018

(54) METHODS OF DIAGNOSING AND TREATING ABIRATERONE ACETATE- GLUCOCORTICOID -RESISTANT OR -SENSITIVE METASTATIC CASTRATION RESISTANT PROSTATE CANCER

(71) Applicant: Janssen Pharmaceutica NV, Beerse (BE)

(72) Inventors: Kazushiro Kawaguchi, Tokyo (JP); Weimin LI, Norristown, PA (US); Ryo Oyama, Tokyo (JP); Jaymala Patel, Bensalem, PA (US); Denis Smirnov, Media, PA (US)

(21) Appl. No.: 15/722,141

(22) Filed: Oct. 2, 2017

Related U.S. Application Data
(60) Provisional application No. 62/402,196, filed on Sep. 30, 2016.

(51) Int. Cl. A61K 31/58; G01N 33/574; A61K 31/573; A61P 35/00

(52) U.S. Cl. CPC A61K 31/58; G01N 33/57434; A61K 31/573; G01N 2333/9108; G01N 2333/948; G01N 2333/90206; G01N 2333/5755; A61P 35/00

(57) ABSTRACT

Disclosed herein are novel biomarkers for detecting resistance and sensitivity to abiraterone acetate-glucocorticoid treatment in a patient having metastatic castration resistant prostate cancer. Also provided are methods of diagnosing and treating abiraterone acetate-glucocorticoid resistant and abiraterone acetate-glucocorticoid sensitive metastatic castration resistant prostate cancer.

### CROSS REFERENCE TO RELATED APPLICATIONS

This application claims priority to U.S. Provisional Application No. 62/402,196, filed Sep. 30, 2016, the disclosure of which is hereby incorporated by reference in its entirety.

### TECHNICAL FIELD

Provided herein are methods of diagnosing and treating abiraterone acetate-glucocorticoid treatment resistant and abiraterone acetate-glucocorticoid treatment sensitive metastatic castration resistant prostate cancer in a patient and biomarkers for diagnosing a patient as having acquired resistance to abiraterone acetate-glucocorticoid treatment.

### BACKGROUND

Prostate cancer is the second most common cancer among men in the United States. It is also one of the leading causes of cancer death among men of all races and Hispanic origin populations. In 2010, 196,038 men in the United States were diagnosed with prostate cancer while 28,560 men in the United States died from prostate cancer. With demographic aging, the Westernization of diet, and advanced diagnostic techniques, the number of patients with prostate cancer in Japan has increased in recent years.

A number of therapeutic agents have been approved by the FDA for use in patients with metastatic castration-resistant prostate cancer (mCRPC). Among these treatment options are docetaxel, abiraterone acetate, cabazitaxel, enzalutamide, mitoxantrone, radium-223, and sipuleucel-T. As a result, clinicians and patients are challenged with a multitude of treatment options and potential sequencing of these agents that make clinical decision-making more complex.

### SUMMARY

Disclosed herein are methods of diagnosing and treating abiraterone acetate-glucocorticoid treatment resistant metastatic castration resistant prostate cancer in a patient comprising, consisting of and/or consisting essentially of, in a patient who has been treated with abiraterone acetate and glucocorticoid, diagnosing the patient as having abiraterone acetate-glucocorticoid treatment resistant metastatic castration resistant prostate cancer when the level of PSMA, ACADL, NPY, UBE2C, or any combination thereof in the biological sample of the patient is elevated, and treating the diagnosed patient with a therapeutic agent other than abiraterone acetate and glucocorticoid or with abiraterone acetate and glucocorticoid in combination with an additional therapeutic agent.

Also provided are methods of diagnosing and treating abiraterone acetate-glucocorticoid sensitive metastatic castration resistant prostate cancer in a patient comprising, consisting of and/or consisting essentially of, in a patient who has been treated with abiraterone acetate and glucocorticoid; diagnosing the patient as having abiraterone acetate-glucocorticoid treatment sensitive metastatic castration resistant prostate cancer when the level of PSMA, ACADL, NPY, UBE2C, or any combination thereof in the biological sample of the patient is not elevated, and treating the diagnosed patient with a therapeutically effective amount of abiraterone acetate and therapeutically effective amount of glucocorticoid.

Methods of detecting resistance or sensitivity to abiraterone acetate-glucocorticoid treatment in a patient having metastatic castration resistant prostate cancer are also provided, the methods comprising, consisting of and/or consisting essentially of determining a level of PSMA, ACADL, NPY, UBE2C, or any combination thereof in a biological sample from the patient who has received abiraterone acetate and glucocorticoid treatment, wherein an elevated level of PSMA, ACADL, NPY, UBE2C, or any combination thereof is indicative of acquired resistance to abiraterone acetate and glucocorticoid treatment and wherein a non-elevated level of PSMA, ACADL, NPY, UBE2C, or any combination thereof is indicative of sensitivity to abiraterone acetate and glucocorticoid treatment.

Disclosed are methods of treating metastatic castration resistant prostate cancer in a patient comprising, consisting of and/or consisting essentially of administering a therapeutically effective amount of abiraterone acetate and a therapeutically effective amount glucocorticoid to the patient, wherein the patient does not have an elevated level of ACADL, NPY, UBE2C, or any combination thereof.

Use of PSMA, ACADL, NPY, UBE2C, or any combination thereof in predicting sensitivity or acquired resistance to abiraterone acetate and glucocorticoid treatment in a patient having metastatic castration resistant prostate cancer is also disclosed.

In the disclosed method and uses, the glucocorticoid may be prednisone, a prednisolone, hydrocortisone, dexamethasone, cortisone, or methylprednisolone.

### BRIEF DESCRIPTION OF THE DRAWINGS

The summary, as well as the following detailed description, is further understood when read in conjunction with the appended drawings. For the purpose of illustrating the disclosed methods, there are shown in the drawings exemplary embodiments of the methods; however, the methods are not limited to the specific embodiments disclosed. In the drawings:

FIG. 1A illustrates gene expression at baseline and EOT for PSMA. FIG. 1B illustrates gene expression at baseline and EOT for full length androgen receptor (AR) and androgen receptor variants (ARVs). Expression (%) is denoted in comparison to 30 healthy subject controls.

FIG. 2A illustrates PSMA gene expression at baseline and at EOS/T and association with secondary endpoints. FIG. 2B illustrates AR and ARV gene expression at baseline and at EOS/T and association with secondary endpoints. Asterisks indicate statistical significance (p<0.05).

FIG. 3A illustrates AR protein expression by CTC counts (AR+ CTC counts) at baseline and EOS/T. FIG. 3B illustrates AR protein expression by CTC counts (AR− CTC counts) at baseline and EOS/T. Connected lines denote paired samples.

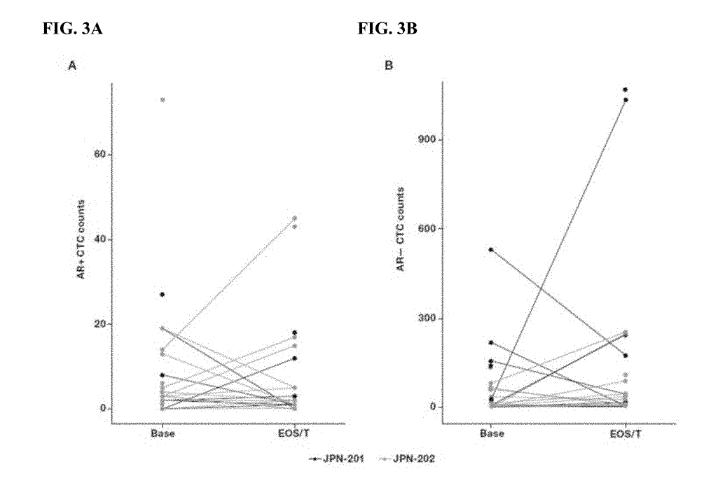

### DETAILED DESCRIPTION OF ILLUSTRATIVE EMBODIMENTS

The disclosed methods may be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods.

Unless specifically stated otherwise, any description as to a possible mechanism or mode of action or reason for improvement is meant to be illustrative only, and the disclosed methods are not to be constrained by the correctness or incorrectness of any such suggested mechanism or mode of action or reason for improvement.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

All ranges are inclusive and combinable. When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

The following abbreviations are used throughout the disclosure: abiraterone acetate (AA); abiraterone acetate plus low-dose prednisone (AA+P); circulating tumor cells (CTC); end of treatment (EOT); end of study (EOS); end of treatment/study (EOT/S); metastatic castration-resistant prostate cancer (mCRPC); overall survival (OS); progression free survival (PFS); and radiographic PFS (rPFS).

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

Abiraterone acetate (“AA”) or ZYTIGA brand abiraterone acetate is a 17a-hydroxylase/C17,20-lyase (CYP17) inhibitor that blocks androgen synthesis in the testes, adrenal gland, and prostate tumor.

The term “comprising” is intended to include examples encompassed by the terms “consisting essentially of” and “consisting of”; similarly, the term “consisting essentially of” is intended to include examples encompassed by the term “consisting of.”

End of treatment (EOT), end of study (EOS), and end of treatment/study (EOT/S) are used interchangeably herein.

As used herein, “therapeutically effective amount” means an amount of a therapeutic agent determined to produce any treatment response in a patient.

As used herein, “treating” means the use of a therapeutic agent for the cure or amelioration of cancer, including prostate cancer.

The disclosed methods are based on the finding that the mRNA level of PSMA, ACADL, NPY, UBE2C, or any combination thereof can be used to diagnose and/or treat abiraterone acetate-glucocorticoid resistant, or abiraterone acetate-glucocorticoid sensitive, metastatic castration resistant prostate cancer (mCRPC) in a patient that has received abiraterone acetate and glucocorticoid treatment. The use of these biomarkers in the diagnosis and treatment of the underlying prostate cancer are non-routine and unconventional.

The methods of diagnosing abiraterone acetate-glucocorticoid resistant, or abiraterone acetate-glucocorticoid sensitive, mCRPC in a patient that has received abiraterone acetate and glucocorticoid treatment comprise:

in a patient who has received abiraterone acetate and glucocorticoid treatment, diagnosing the patient as having abiraterone acetate-glucocorticoid resistant mCRPC when the level of PSMA, ACADL, NPY, UBE2C, or any combination thereof in the biological sample of the patient is elevated; or

diagnosing the patient as having abiraterone acetate-glucocorticoid sensitive mCRPC when the level of PSMA, ACADL, NPY, UBE2C, or any combination thereof in the biological sample of the patients is not elevated.

In some embodiments, the glucocorticoid is prednisone. In some embodiments, the glucocorticoid is a prednisolone. In some embodiments, the glucocorticoid is hydrocortisone. In some embodiments, the glucocorticoid is dexamethasone. In some embodiments, the glucocorticoid is cortisone. In some embodiments, the glucocorticoid is methylprednisolone.

In some embodiments, the methods further comprise, prior to the diagnosing, detecting a level of PSMA, ACADL, NPY, UBE2C, or any combination thereof in a biological sample of the patient.

Also disclosed are methods of treating metastatic castration resistant prostate cancer in a patient. In some embodiments, the methods include administering a therapeutically effective amount of abiraterone acetate and therapeutically effective amount of glucocorticoid to the patient, wherein the patient does not have an elevated level of PSMA, ACADL, NPY, UBE2C, or any combination thereof. Thus, the methods can be used to treat a patient having abiraterone acetate-glucocorticoid sensitive mCRPC. In some embodiments, the methods of treating metastatic castration resistant prostate cancer in a patient include administering a therapeutic agent other than abiraterone acetate and/or glucocorticoid or administering abiraterone acetate and glucocorticoid plus an additional therapeutic agent to the patient, wherein the patient has an elevated level of PSMA, ACADL, NPY, UBE2C, or any combination thereof. Thus, the methods can be used to treat a patient having abiraterone acetate-glucocorticoid resistant mCRPC.

In some embodiments of the methods of treatment, the patient has received abiraterone acetate and glucocorticoid treatment. Thus, the methods of treating mCRPC can include administering a therapeutically effective amount of abiraterone acetate and a therapeutically effective amount of glucocorticoid to a patient who has received abiraterone acetate and glucocorticoid treatment, wherein the patient does not have an elevated level of PSMA, ACADL, NPY, UBE2C, or any combination thereof.

In some of embodiments of the methods of treatment, the glucocorticoid is prednisone. In some embodiments, the glucocorticoid is a prednisolone. In some embodiments, the glucocorticoid is hydrocortisone. In some embodiments, the glucocorticoid is dexamethasone. In some embodiments, the glucocorticoid is cortisone. In some embodiments, the glucocorticoid is methylprednisolone.

In some embodiments of the methods of treatment, the level of PSMA, ACADL, NPY, UBE2C, or any combination thereof is a level of PSMA, ACADL, NPY, or UBE2C mRNA. Thus, the methods include administering a therapeutically effective amount of abiraterone acetate and therapeutically effective amount of glucocorticoid to a patient, wherein the patient does not have an elevated mRNA level of PSMA, ACADL, NPY, UBE2C, or any combination thereof. The level of PSMA, ACADL, NPY, UBE2C, or any combination thereof can be compared to a control. Accordingly, in some aspects, the patient does not have an elevated level of PSMA, ACADL, NPY, UBE2C, or any combination thereof compared to a control. Suitable controls include a previous level of PSMA, ACADL, NPY, UBE2C, or any combination thereof from the patient or a level of PSMA, ACADL, NPY, UBE2C, or any combination thereof in a patient or population of patients that does not have metastatic castration resistant prostate cancer. In some aspects, the control level (previous levels of PSMA, ACADL, NPY, UBE2C, or any combination thereof from the patient, or from a patient or population of patients that does not have mCRPC) is obtained from a blood sample.

The methods of diagnosing and treating can be combined to provide a method of diagnosing and treating abiraterone acetate-glucocorticoid resistant, or abiraterone acetate-glucocorticoid sensitive, mCRPC. The methods can be performed on a patient who has received abiraterone acetate and glucocorticoid treatment. The methods of diagnosing and treating abiraterone acetate-glucocorticoid sensitive metastatic castration resistant prostate cancer in a patient include:

in a patient who has received abiraterone acetate and glucocorticoid treatment, diagnosing the patient as having abiraterone acetate-glucocorticoid sensitive metastatic castration resistant prostate cancer when the level of PSMA, ACADL, NPY, UBE2C, or any combination thereof in the biological sample of the patient is not elevated; and

treating the diagnosed patient with a therapeutically effective amount of abiraterone acetate and a therapeutically effective amount of glucocorticoid.

The methods of diagnosing and treating abiraterone acetate-glucocorticoid resistant metastatic castration resistant prostate cancer in a patient comprise:

in a patient who has received abiraterone acetate and glucocorticoid treatment, diagnosing the patient as having abiraterone acetate-glucocorticoid resistant metastatic castration resistant prostate cancer when the level of PSMA, ACADL, NPY, UBE2C, or any combination thereof in the biological sample of the patient is elevated; and

treating the diagnosed patient with a therapeutic agent other than abiraterone acetate and glucocorticoid or with abiraterone acetate and glucocorticoid plus an additional therapeutic agent.

In some embodiments, the methods of diagnosing and treating abiraterone acetate-glucocorticoid sensitive or resistant metastatic castration resistant prostate cancer further comprise, prior to the diagnosing, detecting a level of PSMA, ACADL, NPY, UBE2C, or any combination thereof in a biological sample of the patient. “Biological sample” includes any sample from a patient that contains, or can contain, one or more of PSMA, ACADL, NPY, or UBE2C. In some embodiments, the biological sample includes circulating tumor cells (CTCs). Characterization of biomarkers on CTCs allows “real time”, non-invasive evaluation of tumor cell dynamics, which may be desirable in the evaluation of mCRPC because the collection of fresh tumor biopsies is not part of the standard of care.

In some embodiments, the level of PSMA, ACADL, NPY, or UBE2C is a level of PSMA, ACADL, NPY, or UBE2C mRNA.

The disclosed methods of diagnosing, or methods of diagnosing and treating, can further include, prior to the diagnosis, comparing the level of PSMA, ACADL, NPY, UBE2C, or any combination thereof to a control. In some embodiments, for example, the methods of diagnosing abiraterone acetate-glucocorticoid resistant, or abiraterone acetate-glucocorticoid sensitive, mCRPC in a patient that has received abiraterone acetate and glucocorticoid treatment include:

comparing a level of PSMA, ACADL, NPY, UBE2C, or any combination thereof in a biological sample of the patient to a control, wherein the patient has received abiraterone acetate and glucocorticoid treatment; and

diagnosing the patient as having abiraterone acetate-glucocorticoid resistant mCRPC when the level of PSMA, ACADL, NPY, UBE2C, or any combination thereof in the biological sample of the patient is elevated; or

diagnosing the patient as having abiraterone acetate-glucocorticoid sensitive mCRPC when the level of PSMA, ACADL, NPY, UBE2C, or any combination thereof in the biological sample of the patient is not elevated.

The method of diagnosing may further comprise, prior to the diagnosing, detecting a level of PSMA, ACADL, NPY, UBE2C, or any combination thereof in a biological sample of the patient, wherein the patient has received abiraterone acetate and glucocorticoid treatment.

In the disclosed methods, the patients can be diagnosed as having abiraterone acetate-glucocorticoid resistant mCRPC when the level of mRNA of PSMA, ACADL, NPY, UBE2C, or any combination thereof in the biological sample of the patient is elevated. In the disclosed methods, the patients can be diagnosed as having abiraterone acetate sensitive mCRPC when the level of mRNA of PSMA, ACADL, NPY, UBE2C, or any combination thereof in the biological sample of the patient is not elevated.

In some embodiments, the methods of diagnosing and treating abiraterone acetate-glucocorticoid sensitive mCRPC in a patient include:

comparing a level of PSMA, ACADL, NPY, UBE2C, or any combination thereof in a biological sample of the patient to a control, wherein the patient has received abiraterone acetate and glucocorticoid treatment;

diagnosing the patient as having abiraterone acetate-glucocorticoid sensitive mCRPC when the level of PSMA, ACADL, NPY, UBE2C, or any combination thereof in the biological sample of the patient is not elevated; and

treating the diagnosed patient with a therapeutically effective amount of abiraterone acetate and a therapeutically effective amount of glucocorticoid.

In other embodiments, the methods of diagnosing and treating abiraterone acetate-glucocorticoid resistant mCRPC in a patient include:

comparing a level of PSMA, ACADL, NPY, UBE2C, or any combination thereof in a biological sample of the patient to a control, wherein the patient has received abiraterone acetate and glucocorticoid treatment;

diagnosing the patient as having abiraterone acetate-glucocorticoid resistant mCRPC when the level of PSMA, ACADL, NPY, UBE2C, or any combination thereof in the biological sample of the patient is elevated; and

treating the diagnosed patient with a therapeutic agent other than abiraterone acetate and/or glucocorticoid or with abiraterone acetate and glucocorticoid plus an additional therapeutic agent.

The method of diagnosing and treating can further comprise, prior to the comparing, detecting a level of PSMA, ACADL, NPY, UBE2C, or any combination thereof in a biological sample of the patient, wherein the patient has received abiraterone acetate and glucocorticoid treatment.

The control can include a previous level of PSMA, ACADL, NPY, UBE2C, or any combination thereof from the patient. In some aspects, the previous level of PSMA, ACADL, NPY, UBE2C, or any combination thereof is from the patient prior to treatment with abiraterone acetate and glucocorticoid. In some aspects, the previous level of PSMA, ACADL, NPY, UBE2C, or any combination thereof is from the patient during treatment with abiraterone acetate and glucocorticoid.

The control can comprise a level of PSMA, ACADL, NPY, UBE2C, or any combination thereof from a patient or population of patients that does not have mCRPC.

Treating the diagnosed patient with a therapeutically effective amount of abiraterone acetate and a therapeutically effective amount glucocorticoid can include the concurrent or sequential administration of abiraterone acetate and glucocorticoid. In some embodiments, the abiraterone acetate and glucocorticoid are co-administered. In some embodiments, the abiraterone acetate and glucocorticoid administered sequentially in either order.

Treating the diagnosed patient with a therapeutic agent other than abiraterone acetate and glucocorticoid or with abiraterone acetate and glucocorticoid plus an additional therapeutic can include treating the patient with a known mCRPC therapy in place of, or in addition to, abiraterone acetate and glucocorticoid.

Also disclosed are methods of detecting resistance or sensitivity to abiraterone acetate and glucocorticoid treatment in a patient having mCRPC, the method includes:

determining a level of PSMA, ACADL, NPY, UBE2C, or any combination thereof in a biological sample from the patient who has received abiraterone acetate and glucocorticoid treatment,

wherein an elevated level of PSMA, ACADL, NPY, UBE2C, or any combination thereof is indicative of acquired resistance to abiraterone acetate-glucocorticoid treatment and

wherein a non-elevated level of PSMA, ACADL, NPY, UBE2C, or any combination thereof is indicative of sensitivity to abiraterone acetate-glucocorticoid treatment.

In some embodiments, the level of PSMA, ACADL, NPY, UBE2C, or any combination thereof is a level of PSMA, ACADL, NPY, or UBE2C mRNA.

In some embodiments of the methods of detecting resistance or sensitivity, the glucocorticoid is prednisone. In some embodiments, the glucocorticoid is a prednisolone. In some embodiments, the glucocorticoid is hydrocortisone. In some embodiments, the glucocorticoid is dexamethasone. In some embodiments, the glucocorticoid is cortisone. In some embodiments, the glucocorticoid is methylprednisolone.

In some embodiments, the patient has an elevated or non-elevated level of PSMA, ACADL, NPY, UBE2C, or any combination thereof compared to a control. The control can include a previous level of PSMA, ACADL, NPY, UBE2C, or any combination thereof from the patient. In some aspects, the previous level of PSMA, ACADL, NPY, UBE2C, or any combination thereof is from the patient prior to treatment with abiraterone acetate and glucocorticoid. In some aspects, the previous level of PSMA, ACADL, NPY, UBE2C, or any combination thereof is from the patient during treatment with abiraterone acetate and glucocorticoid. The control can include a level of PSMA, ACADL, NPY, UBE2C, or any combination thereof from a patient or population of patients that does not have mCRPC.

Also disclosed is the use of PSMA, ACADL, NPY, UBE2C, or any combination thereof in predicting sensitivity or acquired resistance to abiraterone acetate-glucocorticoid therapy in a patient having mCRPC. Further provided is PSMA, ACADL, NPY, UBE2C, or any combination thereof for use in predicting sensitivity or acquired resistance to abiraterone acetate-glucocorticoid treatment in a patient having metastatic castration resistant prostate cancer.

In some embodiments of the disclosed uses, the glucocorticoid is prednisone. In some embodiments, the glucocorticoid is prednisolone. In some embodiments, the glucocorticoid is hydrocortisone. In some embodiments, the glucocorticoid is dexamethasone. In some embodiments, the glucocorticoid is cortisone. In some embodiments, the glucocorticoid is methylprednisolone.

In some embodiments, the PSMA, ACADL, NPY, UBE2C, or any combination thereof is mRNA obtained from a biological sample from the patient, wherein the patient has received abiraterone acetate and glucocorticoid.

The amount of abiraterone acetate that is administered to the patient may be about 250 to about 1500 mg/day, about 500 to about 1000 mg/day, about 500 mg/day, about 750 mg/day or about 1000 mg/day.

The amount of glucocorticoid that is administered to the patient may be about 2.5 to about 15 mg/day, about 5 to about 10 mg/day, about 5 mg/day about 7.5 mg/day, about 10 mg. day, or about 12.5 mg/day. In some embodiments, for example, the glucocorticoid is prednisone, and the amount of prednisone administered is about 2.5 to about 15 mg/day, about 5 to about 10 mg/day, about 5 mg/day about 7.5 mg/day, about 10 mg. day, or about 12.5 mg/day.

In some embodiments, the glucocorticoid may be a prednisolone, hydrocortisone, dexamethasone, cortisone, and methylprednisolone. The amounts of a prednisolone, hydrocortisone, dexamethasone, cortisone, and methylprednisolone may be about 2 to about 10 mg/day, 3 to 6 mg/day, 4 mg/day or 5 mg/day.

### Examples

The following examples are provided to further describe some of the embodiments disclosed herein. The examples are intended to illustrate, not to limit, the disclosed embodiments.

### Study Design and Treatments

Two phase 2 studies, referred to as JPN-201 and JPN-202, were conducted in Japan. JPN-201 and JPN-202 were open-label, multicenter, single-arm, phase 2 studies of 1000 mg abiraterone acetate plus 10 mg prednisone daily (AA) in Japanese patients with chemotherapy-naïve (JPN-201) and chemotherapy-pretreated (JPN-202) mCRPC. Patients were orally given abiraterone acetate (1000 mg q.d.) at least 1 hour before a meal and 2 hours after a meal any time up to 10 pm every day. In addition, 5 mg of oral prednisolone was concomitantly administered twice a day. A 28-daily dosing cycle continued until disease progression or unacceptable toxicity was observed.

The primary end point was the proportion of patients achieving PSA response (i.e., a≧50% PSA decline from baseline) by 12 weeks of therapy in accordance with Prostate-Specific Antigen Working Group (PSAWG) criteria. Secondary end points included:

PSA response rate (confirmed or unconfirmed) during the treatment period;

Overall survival (OS);

PSA-PFS based on progression defined in accordance with PSAWG criteria;

Radiographic PFS (rPFS) based on progression defined by RECIST Version 1.0; and

Modified PFS.

Patients were required to have a PSA level of ≧5 ng/mL and an Eastern Cooperative Oncology Group performance status (ECOG PS) score of 0 or 1. Patients received study treatment until disease progression or unacceptable toxicity unless the investigator deemed that they continued to derive clinical benefit. The review boards at all participating institutions approved the study, which was conducted according to the Declaration of Helsinki. All patients provided written, informed consent to participate in the study.

### Biomarker Analysis

An exploratory biomarker analysis was conducted on patient data from JPN-201 and JPN-202 to explore the potential difference in efficacy by comparing PSA response and measures of survival between the two studies and to explore the molecular mechanism of abiraterone resistance in the combined Japanese patient population with mCRPC. Several biomarkers associated with the mechanism of action of abiraterone or with the development and progression of prostate cancer were evaluated in this analysis (Table 1).

TABLE 1
Biomarkers evaluated

| Biomarker | Analysis | Time point |
|---|---|---|
| CTC | Enumeration^{a} | Baseline, C2D1, C3D1, |
| | | C4D1, and EOT |
| AR expression^{b} | CellSearch ® CXC Kit^{c} | Baseline and EOT |
| mRNA biomarker | RT-PCR | Baseline and EOT |
| expression^{d} | | |

^{a}CTCs were stratified as ≥5 or <5 CTCs.
^{b}AR nuclear protein expression was dichotomized as <10% or ≥10% in CTCs.
^{c}Determination of AR protein expression was conducted using CellSearch ® CXC Kit with a phycoerythrin-labeled monoclonal antibody for AR protein expression detection.
^{d}Additional mRNA biomarkers evaluated, including AR FL, AR splice variants, KLK3 (PSA), PSMA, CYP17, IFIH1, CDH1, were dichotomized as positive or negative expression based on cutoffs established from normal healthy patients (maximum expression in blood samples from normal healthy patients as cutoffs).
Baseline is defined as C1D1.
AR (androgen receptor);
CTC (circulating tumor cell);
C2D1 (cycle 2 day 1);
EOT (end of treatment).

Identification and association of biomarkers with clinical outcomes taken at baseline and at the end of the study or progression was evaluated in each study and compared across the studies. An association analysis of biomarkers with clinical end points was performed initially with the primary study end points. If positive associations were found, then additional association analyses of secondary end points were performed. Molecular characteristics of the subset of patients who did not respond to abiraterone treatment were evaluated, compared with those with good response, to identify biomarker profiles that correlate with resistance to abiraterone treatment.

Samples were collected from study patients at various time points shown in Table 1. Blood samples (10 mL) were collected for CTC enumeration on Day 1 from Cycle 1 (baseline) through Cycle 4 and end of treatment (EOT). An additional blood sample was collected for determination of AR protein expression at Cycle 1 Day 1 (baseline) and end of treatment (EOT). An EDTA blood sample was collected at baseline and EOT for analysis of RNA.

### CTC Enumeration

CTC enumeration was performed at Clinical Research Solutions (CRS) laboratory (Janssen Diagnostics, LLC, Huntingdon Valley, Pa.). The number of CTC was determined using the CellSearch® CTC Kit (Catalog No. 7900001) following the manufacturer's instructions. Samples were processed in the CellTracks® AutoPrep® system and analyzed in CellTracks® Analyzer II.

### AR Protein Expression

AR protein expression analysis (CTC nuclei staining) was conducted at CRS laboratory using the CellSearch® CXC Kit (Catalog No. 7900017) following the manufacturer's instructions. CTC were stained with phycoerythrin-labeled monoclonal antibody for detection of AR protein expression. The normal blood spiked with the LNCaP cells processed without the AR marker reagent (background control) and in the presence of AR marker reagent (positive control) was used to establish the background signal in the phycoerythrin channel and a control for the reagent, respectively.

### Gene Expression Analysis

A gene expression assay panel of mRNA biomarkers from predefined RNA groups was developed using quantitative RT-PCR. Biomarker analysis was performed as single-marker analysis. Assays were developed to detect AR full length (AR FL) and AR splicing variants that are missing ligand binding domain (ARV1, ARV7, and ARV567), GAPDH and RPL19 (universal controls), PSMA, CDH1, IFIH1, NPY, UBE2C and ACADL, using prostate model cell line (LNCaP) spiked (0, 10, 50, and 100 cells) in normal donor blood.

Blood samples from clinical study patients were collected and shipped to a contracted site of Janssen Pharmaceutical KK, Japan, for RNA sample preparation. RNA was extracted from the frozen cell lysate samples from the clinical study patients using Qiagen RNeasy® Plus Micro kit (Catalog No. 74034) following the manufacturer's instructions, followed by cDNA synthesis using High Capacity cDNA Reverse Transcription kit (Thermo Fisher Scientific, Catalog No. 4368814) with RNAse inhibitor, following the manufacturer's instructions. Pre-amplification was performed using Taqman Pre-amp mastermix (Thermo Fisher Scientific, Catalog No. 4391128) following the manufacturer's instructions, and then PCR analysis was performed using ABI (Applied Biosystems) Taqman primer/Probes pairs. RNA samples were analyzed and normalized using RPL19 (housekeeping gene; NCBI Gene ID: 6143), with positive gene expression defined as higher than the maximum expression observed in healthy controls (59 samples) (i.e. mRNA expression higher than the maximum expression observed in non-mCRPC patients was regarded as positive gene expression).

### Patients

Ninety-five patients (48 chemotherapy-naïve patients and 47 chemotherapy-pretreated patients in JPN-201 and JPN-202, respectively) were enrolled and treated between both trials. The biomarker analysis included all patients treated on study who had data from at least one of the clinical end points and had evaluable biomarker data (Table 2). All patients from JPN-201 and JPN 202 were eligible for the biomarker analysis data set. Biomarkers were excluded from the analysis based on the following preplanned criteria:
CTC protein expression and that had less than 3 CTCs evaluable for a CTC sample were considered not evaluable and removed from the analysis; and
For all CTC mRNA biomarkers, if any of GAPDH or PSA measurements were negative, the sample was regarded as not evaluable for all mRNA biomarkers tested and was excluded from the analysis.

TABLE 2
Patient samples available for biomarker analysis

| | JPN-201 | JPN-202 | Total |
|---|---|---|---|
| Randomized and treated patients, n | 48 | 47 | 95 |
| Baseline | | | |
| # of blood samples, n | 48 | 47 | 95 |
| CTC enumeration | 48 | 47 | 95 |
| RT-PCR | 46 | 47 | 93 |
| EOT | | | |
| # of blood samples, n | 43 | 31 | 74 |
| CTC enumeration | 36 | 25 | 61 |
| RT-PCR | 35 | 24 | 59^{a} |
| Baseline and EOT | | | |
| CTC enumeration | 36 | 25 | 61 |
| RT-PCR | 34 | 24 | 58 |

^{a}59 healthy patient control samples were also available for mRNA biomarker analysis

The data from patients who experienced serious adverse events leading to discontinuation of treatment were removed from the analysis. Provided below is an analysis of those patients that remained on treatment.

The paired McNemar test was used to evaluate the association of dichotomized categorical biomarker changes from baseline to EOT, and the paired Wilcoxon rank sum test was used to evaluate continuous biomarker changes from baseline to EOT. Association analysis of biomarkers and change of biomarkers with clinical response in the JPN-201 and JPN-202 studies combined and in each study separately was analyzed. Logistic regression or Fisher's exact test were used to evaluate association with biomarker levels at baseline or EOT with clinical endpoints, or the association with change of biomarker levels at EOT from baseline with PSA response. CTC counts were included in the gene expression association analysis with clinical outcomes to correct for variability in CTCs between patients. Cox proportional hazard model with CTC number correction was used to evaluate the association of biomarker levels with time to event end points, overall survival, rPFS and PSA-based PFS. Survival analysis was performed for time to event endpoints with correction for CTCs. Unadjusted P values were reported for all analyses.

The treatment effect, biomarker expression EOT versus baseline, is provided in Table 3 below.

TABLE 3
Baseline versus end of treatment biomarker gene expression

| | | EOT | | % Change | |
|---|---|---|---|---|---|
| Biomarker | Baseline | Negative | Positive | from baseline | p Value |
| AR FL | Positive | 8 | 21 | 2% | 1.000 |
| | Negative | 20 | 9 | | |
| ARV1 | Positive | 4 | 2 | 29% | 0.001 |
| | Negative | 31 | 21 | | |
| ARV7 | Positive | 4 | 11 | 22% | 0.009 |
| | Negative | 26 | 17 | | |
| PSMA | Positive | 0 | 25 | 21% | 0.003 |
| | Negative | 16 | 11 | | |
| CDH1 | Positive | 3 | 15 | 12% | 0.149 |
| | Negative | 25 | 9 | | |
| IFIH1 | Positive | 3 | 1 | −2% | 1.000 |
| | Negative | 46 | 2 | | |

Gene expression was dichotomized using maximum levels observed in healthy controls.
Base levels in rows, EOT levels in columns.
p-values are unadjusted p-values.

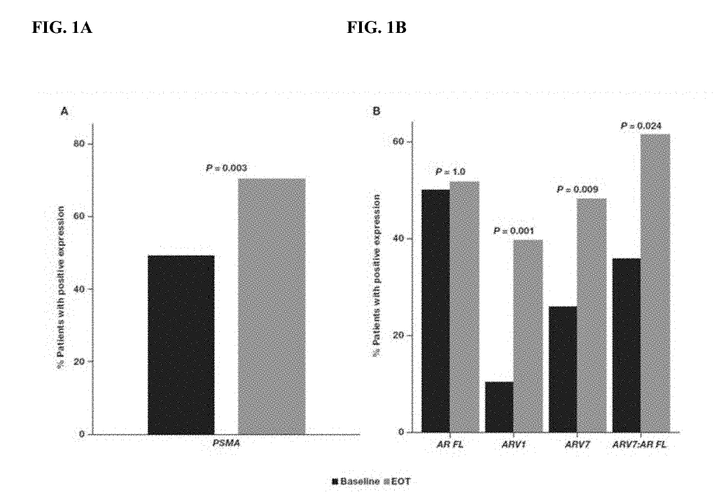

Changes in gene expression from baseline to EOT/EOS were analyzed, and results for genes of interest are presented in FIG. 1A, FIG. 1B, and Table 3. The percentage of patients with PSMA expression was higher at EOT versus baseline in the combined study analysis (69% vs. 48%, P=0.003; FIG. 1A) No significant change in the percentage of patients with AR FL expression was observed from baseline (52% vs. 50%, P=1.0), while patients with ARV 1 expression and ARV7 expression were higher at EOT versus baseline (40% vs. 10% and 48% vs. 26%, respectively, P<0.01) (FIG. 1B). Baseline and EOT expression of ARV567 was not detectable in the majority of patient samples (data not shown).

Association of baseline biomarkers with clinical response and resistance was evaluated. Table 4 shows the primary efficacy outcomes (PSA response) at baseline and Table 5 shows the PSA response at EOT.

TABLE 4
Association analysis of baseline biomarkers with PSA response
for the JPN-201 and JPN-202 studies combined

| | Non Resp^{a} | Resp^{a} | Total | Odds ratio | |
|---|---|---|---|---|---|
| Biomarker | n (%) | n (%) | n (%) | (95% CI) | p Value |
| ARV7 | | | | | |
| expression^{b} | | | | | |
| Negative | 28 (74) | 25 (76) | 53 (76) | 0.897 | 1.000 |
| Positive | 10 (26) | 8 (24) | 18 (25) | (0.262-2.987) | |
| ARV1 | | | | | |
| expression^{b} | | | | | |
| Negative | 32 (84) | 29 (88) | 61 (86) | 0.739 | 0.742 |
| Positive | 6 (16) | 4 (12) | 10 (14) | (0.139-3.480) | |
| AR full length | | | | | |
| expression^{b} | | | | | |
| Negative | 14 (37) | 19 (58) | 33 (46) | 0.435 | 0.099 |
| Positive | 24 (63) | 14 (42) | 38 (54) | (0.148-1.235) | |
| PSMA | | | | | |
| expression^{b} | | | | | |
| Negative | 12 (36) | 20 (69) | 32 (52) | 0.263 | 0.013 |
| Positive | 21 (64) | 9 (31) | 30 (48) | (0.078-0.833) | |

^{a}PSA response is defined as ≥50% PSA decline by 12 weeks.
^{b}Gene expression has been dichotomized per gene using the maximum level observed in healthy controls, with all RNA samples regardless of CTC count included.
Frequencies as column-wise percentages are shown.
P values are based on Fisher's exact test.

TABLE 5
Association analysis of EOT biomarkers with PSA response for the
JPN-201 and JPN-202 studies combined

| | Non Resp^{a} | Resp^{a} | Total | Odds ratio | |
|---|---|---|---|---|---|
| Biomarker | n (%) | n (%) | n (%) | (95% CI) | p Value |
| ARV7 | | | | | |
| expression^{b} | | | | | |
| Negative | 11(35) | 18(69) | 29(51) | 0.251 | 0.017 |
| Positive | 20(65) | 8(31) | 28(49) | (0.069, 0.842) | |
| ARV1 | | | | | |
| expression^{b} | | | | | |
| Negative | 15(48) | 19(73) | 34(60) | 0.352 | 0.103 |
| Positive | 16(52) | 7(27) | 23(40) | (0.096, 1.193) | |
| ARV total | | | | | |
| expression^{c} | | | | | |
| Negative | 9(29) | 17(65) | 26(46) | 0.223 | 0.008 |
| Positive | 22(71) | 9(35) | 31(54) | (0.061, 0.755) | |
| PSMA | | | | | |
| expression^{b} | | | | | |
| Negative | 4(14) | 12(52) | 16(31) | 0.153 | 0.006 |
| Positive | 25(86) | 11(48) | 36(69) | (0.029, 0.646) | |
| NPY | | | | | |
| expression^{b} | | | | | |
| Negative | 12(41) | 18(78) | 30(58) | 0.203 | 0.011 |
| Positive | 17(59) | 5(22) | 22(42) | (0.045, 0.772) | |
| ACADL | | | | | |
| expression^{b} | | | | | |
| Negative | 14(48) | 16(70) | 30(58) | 0.416 | 0.162 |
| Positive | 15(52) | 7(30) | 22(42) | (0.109, 1.471) | |
| UBE2C | | | | | |
| expression^{b} | | | | | |
| Negative | 26(90) | 22(96) | 48(92) | 0.4 | 0.621 |
| Positive | 3(10) | 1(4) | 4(8) | (0.007, 5.402) | |

^{a}PSA response is defined as ≥50% PSA decline by 12 weeks.
^{b}Gene expression has been dichotomized per gene using the maximum level observed in healthy controls, with all RNA samples regardless of CTC count included.
^{c}Total AR expression is defined as positive expression for any one of ARV1, ARV7 or ARV567.
Frequencies as column-wise percentages are shown.
P values are based on Fisher's exact test.

With regard to gene expression associations with the primary endpoint, baseline and EOT PSMA expression was associated with lower PSA response rates (baseline: 30% in PSMA+vs. 63% in PSMA−, P=0.01; EOT: 31% in PSMA+vs. 75% in PSMA−, P=0.01). Baseline and EOT AR FL expression did not have a significant effect on PSA response (37% in AR FL+vs. 58% in AR FL−, P=0.10 and 43% in AR FL+vs. 48% in AR FL−, P=0.79, respectively). Baseline ARV1 or ARV7 expression did not have a significant effect on PSA response (40% in ARV1+vs. 48% in ARV1−, P=0.74; 44% in ARV7+vs. 47% in ARV7−, P=1.0). EOT ARV7 expression was associated with lower PSA response (29% in ARV7+vs. 62% in ARV7−, P≦0.02). Positive EOT NPY expression was associated with lower PSA response rate (22% responders vs 59% nonresponders, p=0.011).

Of the baseline patient samples collected for the biomarker analysis, 44% had ≧5 CTCs (Table 6). Of patients with baseline CTC≧5, 34% to 54% converted to CTC<5 on treatment and 20% maintained CTC<5 at EOT/EOS. Most samples with baseline CTC<5 (56% at baseline) maintained <5 on treatment (89-94%) until progression (41% EOT/EOS).

TABLE 6
Number (percentage) of patients in CTC conversion classes at each
time point compared with baseline for JPN-201 and JPN-202

| CTC | | | | | EOT or |
|---|---|---|---|---|---|
| category | C1D1 | C2D1 <5 | C3D1 <5 | C4D1 <5 | EOS <5 |
| <5 | 38/68 (56) | 33/35 (94) | 36/37 (97) | 32/36 (89) | 11/27 (41) |
| ≥5 | 30/68 (44) | 10/29 (34) | 12/29 (41) | 14/26 (54) | 4/20 (20) |
| Total <5 | 38/68 (56) | 43/64 (67) | 48/66 (73) | 46/62 (74) | 15/47 (32) |

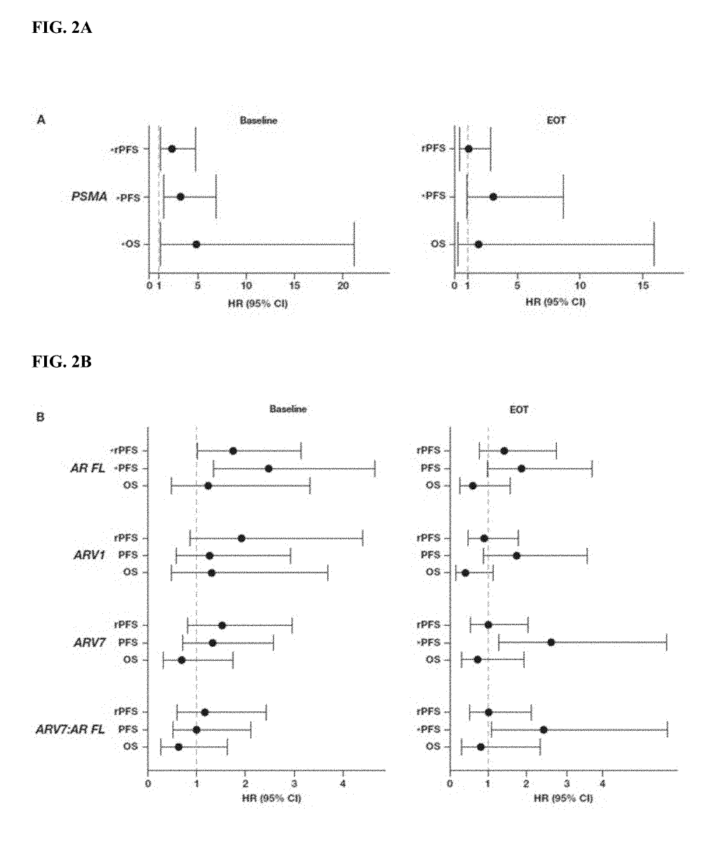

With regard to gene expression associations with secondary endpoints (FIG. 2A and FIG. 2B), baseline PSMA expression was associated with shorter rPFS, PFS, and OS (HR, 2.3, 3.2, and 4.9, respectively, P<0.05) (FIG. 2A). EOT PSMA expression was associated with shorter PFS (HR, 3.1, P=0.04) (FIG. 2A). Baseline AR FL expression was associated with shorter PFS (HR, 2.5, P=0.004) and shorter rPFS (HR, 1.77, P=0.048; FIG. 2B). No evidence of a significant association between EOT AR FL expression and rPFS, PFS, or OS (HR, 1.46, P=0.25; HR, 1.90, P=0.06; and HR, 0.63, P=0.32, respectively) was observed (FIG. 2B). Baseline ARV7 expression showed no significant association with rPFS, PFS, or OS, while EOT ARV7 expression showed a significant association with shorter PFS only (HR, 2.7, P=0.01; FIG. 2B). EOT expression of CDH1 was associated with shorter PFS (HR, 2.32, P=0.04) and IFIH1 with shorter rPFS (HR, 4.54, P=0.06) (Table 7). Positive EOT expression of UBE2C (proliferation marker) was associated with shorter OS (HR, 3.70, P=0.03) and ACADL (androgen-related fatty acid β-oxidation enzyme) with shorter PFS (HR, 2.48, P=0.02) (Table 7).

TABLE 7
EOT gene expression association with secondary clinical endpoints

| | OS | | PFS | | rPFS | |
|---|---|---|---|---|---|---|
| Biomarker^{a} | HR (95% CI) | P value | HR (95% CI) | P value | HR (95% CI) | P value^{b} |
| CDH1 | 0.69 | 0.50 | 2.32 | 0.04 | 1.41 | 0.36 |
| | (0.23-2.06) | | (1.03-5.23) | | (0.68-2.91) | |
| IF1H1 | 1.25 | 0.83 | 0.46 | 0.30 | 4.54 | 0.06 |
| | (0.16-9.66) | | (0.11-1.98) | | (0.94-22.07) | |
| UBE2C | 3.70 | 0.03 | 1.25 | 0.70 | 1.87 | 0.28 |
| | (1.17-11.68) | | (0.40-3.93) | | (0.60-5.78) | |
| NPY | 0.85 | 0.78 | 1.03 | 0.94 | 0.65 | 0.25 |
| | (0.26-2.76) | | (0.50-2.11) | | (0.31-1.35) | |
| ACADL | 0.73 | 0.56 | 2.48 | 0.02 | 1.11 | 0.75 |
| | (0.26-2.08) | | (1.17-5.29) | | (0.57-2.20) | |

^{a}Genes evaluated were dichotomized as positive or negative expression based on cutoffs established from normal healthy subjects.
^{b}P values are based on the Fisher's exact test.

No significant change in AR protein expression was observed at EOT versus baseline in the combined study analysis (Table 8) and there was no significant change (baseline vs. EOT) in absolute counts of AR+ and AR− CTCs. Discordance between AR FL+ gene expression and AR+ protein expression (Table 9) was observed; 16 of 18 (89%) patient samples with AR+ CTCs were AR FL+ while 10 of 12 (83%) patient samples with AR− CTCs were AR FL+. Positive baseline and EOT AR expression did not have a significant effect on PSA response (37% in AR+vs. 36% in AR−, P=1.0 and 36% in AR+vs. 37% in AR−, P=1.0, respectively) (Table 9). Neither baseline nor EOT AR expression was significantly associated with secondary clinical endpoints (P>0.05); however, a trend of association of baseline AR expression with improvement in rPFS in AA plus prednisone-treated patients was observed (HR, 0.56, P=0.17; Table 10).

TABLE 8
Change of CTC counts from baseline to EOT in AR+ and AR− samples

| | Baseline (n = 31) | EOT (n = 33) | |
|---|---|---|---|
| Biomarker^{a} | Median (range) | Median (range) | P value^{b} |
| AR+ | 3 (0-73) | 1 (0-45) | 0.88 |
| AR− | 7 (0-530) | 17 (3-1069) | 0.47 |

^{a}AR protein expression was dichotomized as <10% (negative) or ≥10% (positive) in CTCs.
Unpaired expression data are included.
^{b}P values are based on the paired Wilcoxon rank sum test.

TABLE 9
Comparison of positive AR protein expression and
gene expression at baseline

| | AR gene expression^{a} | |
|---|---|---|
| AR protein expression^{b} | Positive | Negative |
| Positive | 16 | 2 |
| Negative | 10 | 2 |

^{a}Genes evaluated were dichotomized as positive or negative expression based on cutoffsestablished from normal healthy subjects.
^{b}AR protein expression was dichotomized as <10% (negative) or ≥10% (positive) in CTCs.
Patients with <5 CTCs were removed from the analysis.

TABLE 10
Baseline and EOT AR protein expression association with secondary clinical endpoints

| | OS | | PFS | | rPFS | |
|---|---|---|---|---|---|---|
| Biomarker^{a} | HR (95% CI) | P value^{b} | HR (95% CI) | P value^{b} | HR (95% CI) | P value^{b} |
| AR (BL) | 0.70 | 0.46 | 0.90 | 0.79 | 0.56 | 0.17 |
| | (0.27-1.81) | | (0.42-1.92) | | (0.24-1.28) | |
| AR (EOT) | 0.62 | 0.32 | 1.22 | 0.62 | 0.92 | 0.82 |
| | (0.24-1.59) | | (0.56-2.65) | | (0.44-1.91) | |

^{a}AR protein expression was dichotomized as <10% or ≥10% in CTCs. Patients with >5 CTCs were removed from the analysis.
^{b}P values are based on cox proportional hazard with CTC as a covariate.

### Discussion

The overall goal of the biomarker strategy was to identify biomarker profiles that are associated with sensitivity or resistance to AA plus prednisone in mCRPC in Japanese patients. As treatment selection following AA plus prednisone resistance is challenging, this analysis may provide a biomarker guide to choosing optimal therapy for specific patients based on their molecular profile. This biomarker analysis also has broader implications in clinical practice in that it provides a feasible approach to performing a comprehensive AR axis-centered CTC biomarker study and establishes, for the first time, a method for evaluating AR and its splice variants in CTCs.

Baseline and EOT PSMA expression were identified as a potential biomarker associated with worse outcomes in patients with mCRPC. Positive PSMA expression was detected more frequently in nonresponders for the combined mCRPC patient population and for chemotherapy-naïve patients. In addition, associations between positive baseline PSMA expression and shorter PFS and rPFS were observed for chemotherapy-naïve patients. PSMA expression as a biomarker has relevancy for post-AA plus prednisone therapeutic decision-making and the design of therapeutic strategies for patients with mCRPC, especially for those who have not had prior chemotherapy.

While EOT ARV7 expression was associated with a lower PSA response (29% in ARV7+vs. 62% in ARV7−, P≦0.02) and shorter PFS (HR, 2.7, P=0.01), baseline expression of ARV7 did not show association with clinical outcomes. Positive AR nuclear staining in CTC or expression of wild type AR or AR splice variants did not show association with PSA response, suggesting existence of primary resistance mechanisms to AA and heterogeneity of AR signaling pathways. These results conflict with recent reports that ARV7 expression from patients with castration-resistant prostate cancer may be associated with resistance to abiraterone (Antonarakis E. S., et al. AR-V7 and resistance to enzalutamide and abiraterone in prostate cancer. N Engl J Med 2014; 371:1028-1038; Qu F, et al. Association of AR-V7 and prostate specific antigen RNA levels in blood with efficacy of abiraterone acetate and enzalutamide treatment in men with prostate cancer. Clin Cancer Res 2017; 23:726-34; Del Re M., et al. The detection of androgen receptor splice variant 7 in plasma-derived exosomal RNA strongly predicts resistance to hormonal therapy in metastatic prostate cancer patients. Eur Urol 2017; 71:680-7; and Todenhofer T, et al. AR-V7 transcripts in whole blood RNA of patients with metastatic castration resistant prostate cancer correlate with response to abiraterone acetate. J Urol 2017; 197:135-42). Among men receiving abiraterone acetate, ARV7-positive patients had lower PSA response rates than ARV7-negative patients (0% vs 68%, p=0.004) and shorter PSA-PFS (median, 1.3 months vs not reached; p<0.001), clinical or radiographic progression-free survival (median, 2.3 months vs not reached; p<0.001), and overall survival (median, 10.6 months vs not reached, p=0.006). High baseline ARV7 expression in the blood was also reported to predict worse OS outcomes in AA-treated patients with mCRPC. In a study that evaluated a novel RNA extraction method from plasma-derived exosomes, OS was significantly shorter in ARV7+ participants at baseline compared with ARV7-patients with CRPC treated with AA or enzalutamide (8 months vs. not reached, P<0.001). However, a study has also shown that particular subgroups of patients with CRPC can benefit from AA plus prednisone and/or enzalutamide despite detection of ARV7 splice variants in their CTTCs (Bernemann C, et al. Expression of AR-V7 in circulating tumour cells does not preclude response to next generation androgen deprivation therapy in patients with castration resistant prostate cancer. Eur Urol 2017; 71:1-3). Higher frequencies of patients with positive PSMA expression in PSA nonresponders than in PSA responders were observed when the two studies were combined.

Discordance was observed between the mRNA and protein AR expression and the association analyses between these biomarker and clinical outcomes. In this analysis the mRNA assay appears to have higher sensitivity than the protein assay. Baseline AR FL expression was correlated with worse clinical outcomes, notably a significant association with shorter PFS (HR, 2.5, P=0.004) in AA plus prednisone-treated patients, while baseline AR protein expression showed a trend for improved clinical outcomes, specifically a trend for improved rPFS (HR, 0.56, P=0.17). Inconsistencies in these results may be due to the dependence of AR expression for clinical benefit of AA plus prednisone, as AR FL expression in patients in this analysis did not correlate with AR expression. AA suppressed the expression of genes (e.g., IFIH1, CDH1) typically decreased in docetaxel-resistant tumors. These genes were identified by an exploratory microarray analysis for resistance to docetaxel in two CRPC cell lines (22). The epithelial cell adhesion molecule CDH1 is related to the epithelial-mesenchymal transition process, while IFIH1 has been associated while antiviral cellular responses (22).

### Conclusions

Higher frequency of baseline and EOT PSMA was associated with worse outcomes (i.e. resistance biomarker for AA in patients with mCRPC, especially for chemotherapy-naïve mCRPC patients). Baseline and EOT ARv7 showed a weak association with outcomes. EOT expression of NPY, UBE2C, and ACADL was associated with worse outcomes. AA suppressed expression of genes IFIH1 and CDH1, typically decreased in docetaxel-resistant tumors.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### Embodiments

The following list of embodiments is intended to complement, rather than displace or supersede, the previous descriptions.
Embodiment 1. A method of diagnosing and treating abiraterone acetate-glucocorticoid resistant metastatic castration resistant prostate cancer in a patient comprising:
   in a patient who has received abiraterone acetate and glucocorticoid treatment, diagnosing the patient as having abiraterone acetate-glucocorticoid resistant metastatic castration resistant prostate cancer when a level of PSMA, ACADL, NPY, UBE2C, or any combination thereof in a biological sample of the patient is elevated; and
   treating the diagnosed patient with a therapeutically effective amount of a therapeutic agent other than abiraterone acetate and glucocorticoid or with abiraterone acetate and glucocorticoid plus a therapeutically effective amount of an additional therapeutic agent.
Embodiment 2. A method of diagnosing and treating abiraterone acetate-glucocorticoid sensitive metastatic castration resistant prostate cancer in a patient comprising:
   in a patient who has received abiraterone acetate and glucocorticoid treatment, diagnosing the patient as having abiraterone acetate-glucocorticoid sensitive metastatic castration resistant prostate cancer when a level of PSMA, ACADL, NPY, UBE2C, or any combination thereof in a biological sample of the patient is not elevated; and
   treating the diagnosed patient with a therapeutically effective amount of abiraterone acetate and glucocorticoid treatment.
Embodiment 3. The method of embodiment 1 or 2, further comprising, prior to the diagnosing, detecting a level of PSMA, ACADL, NPY, UBE2C, or any combination thereof in a biological sample of the patient.
Embodiment 4. The method of embodiment 3, wherein the biological sample comprises circulating tumor cells.
Embodiment 5. The method of any one of the previous embodiments, wherein the level of PSMA, ACADL, NPY, or UBE2C is a level of PSMA, ACADL, NPY, or UBE2C mRNA.
Embodiment 6. The method of any one of the previous embodiments, further comprising, prior to the diagnosis, comparing the level of PSMA, ACADL, NPY, UBE2C, or any combination thereof to a control.
Embodiment 7. The method of embodiment 6, wherein the control comprises a previous level of PSMA, ACADL, NPY, UBE2C, or any combination thereof from the patient.
Embodiment 8. The method of embodiment 6, wherein the control comprises a level of PSMA, ACADL, NPY, UBE2C, or any combination thereof from a patient or population of patients that does not have metastatic castration resistant prostate cancer.
Embodiment 9. A method of detecting resistance or sensitivity to abiraterone acetate and glucocorticoid treatment in a patient having metastatic castration resistant prostate cancer, the method comprising:
   determining a level of PSMA, ACADL, NPY, UBE2C, or any combination thereof in a biological sample from the patient who has received abiraterone acetate and glucocorticoid treatment,
   wherein an elevated level of PSMA, ACADL, NPY, UBE2C, or any combination thereof is indicative of acquired resistance to abiraterone acetate-glucocorticoid treatment and
   wherein a non-elevated level of PSMA, ACADL, NPY, UBE2C, or any combination thereof is indicative of sensitivity to abiraterone acetate-glucocorticoid treatment.
Embodiment 10. The method of embodiment 9, wherein the level of PSMA, ACADL, NPY, UBE2C, or any combination thereof is a level of PSMA, ACADL, NPY, or UBE2C mRNA.
Embodiment 11. The method of embodiment 9 or 10, wherein the patient has an elevated or non-elevated level of PSMA, ACADL, NPY, UBE2C, or any combination thereof compared to a control.
Embodiment 12. The method of embodiment 11, wherein the control comprises a previous level of PSMA, ACADL, NPY, UBE2C, or any combination thereof from the patient.
Embodiment 13. The method of embodiment 11, wherein the control comprises a level of PSMA, ACADL, NPY, UBE2C, or any combination thereof in a patient or population of patients that does not have metastatic castration resistant prostate cancer.
Embodiment 14. A method of treating metastatic castration resistant prostate cancer comprising:
   administering a therapeutically effective amount of abiraterone acetate and a therapeutically effective amount of glucocorticoid to a patient having mCRPC, wherein the patient does not have an elevated level of ACADL, NPY, UBE2C, or any combination thereof.
Embodiment 15. The method of embodiment 14, wherein the patient has received abiraterone acetate and glucocorticoid treatment.
Embodiment 16. The method of embodiment 15, wherein the patient does not have an elevated level of PSMA.
Embodiment 17. The method of any one of embodiments 14-16, wherein abiraterone acetate and glucocorticoid are administered concurrently or sequentially.
Embodiment 18. The method of any one of embodiments 14-17, wherein the level of PSMA, ACADL, NPY, UBE2C, or any combination thereof is a level of PSMA, ACADL, NPY, or UBE2C mRNA.
Embodiment 19. The method of any one of embodiments 14-18, wherein the patient does not have an elevated level of PSMA, ACADL, NPY, UBE2C, or any combination thereof compared to a control.
Embodiment 20. The method of embodiment 19, wherein the control comprises a previous level of PSMA, ACADL, NPY, UBE2C, or any combination thereof from the patient.
Embodiment 21. The method of embodiment 20, wherein the control comprises a level of PSMA, ACADL, NPY, UBE2C, or any combination thereof in a patient or population of patients that does not have metastatic castration resistant prostate cancer.
Embodiment 22. A method of treating metastatic castration resistant prostate cancer comprising:
   administering a therapeutically effective amount of abiraterone acetate and a therapeutically effective amount of glucocorticoid to a patient who has received abiraterone acetate and glucocorticoid treatment, wherein the patient does not have an elevated level of PSMA, ACADL, NPY, UBE2C, or any combination thereof.
Embodiment 23. The method of embodiment 22, wherein abiraterone acetate and glucocorticoid are administered concurrently or sequentially.
Embodiment 24. The method of embodiment 22 or 23, wherein the level of PSMA, ACADL, NPY, UBE2C, or any combination thereof is a level of PSMA, ACADL, NPY, or UBE2C mRNA.
Embodiment 25. The method of any one of embodiments 22-24, wherein the patient does not have an elevated level of PSMA, ACADL, NPY, UBE2C, or any combination thereof compared to a control.
Embodiment 26. The method of embodiment 25, wherein the control comprises a previous level of PSMA, ACADL, NPY, UBE2C, or any combination thereof from the patient.
Embodiment 27. The method of embodiment 25, wherein the control comprises a level of PSMA, ACADL, NPY, UBE2C, or any combination thereof in a patient or population of patients that does not have metastatic castration resistant prostate cancer.
Embodiment 28. A method of treating metastatic castration resistant prostate cancer comprising:
   to a patient not having an elevated level of ACADL, NPY, UBE2C, or any combination thereof, administering a therapeutically effective amount of abiraterone acetate and a therapeutically effective amount of glucocorticoid to treat the metastatic castration resistant prostate cancer.
Embodiment 29. The method of embodiment 28, wherein the patient has received abiraterone acetate and glucocorticoid treatment.
Embodiment 30. The method of embodiment 29, wherein the patient does not have an elevated level of PSMA.
Embodiment 31. The method of any one of embodiments 28-30, wherein abiraterone acetate and glucocorticoid are administered concurrently or sequentially.
Embodiment 32. The method of any one of embodiments 28-31, wherein the level of PSMA, ACADL, NPY, UBE2C, or any combination thereof is a level of PSMA, ACADL, NPY, or UBE2C mRNA.
Embodiment 33. The method of any one of embodiments 28-32, wherein the patient does not have an elevated level of PSMA, ACADL, NPY, UBE2C, or any combination thereof compared to a control.
Embodiment 34. The method of embodiment 33, wherein the control comprises a previous level of PSMA, ACADL, NPY, UBE2C, or any combination thereof from the patient.
Embodiment 35. The method of embodiment 34, wherein the control comprises a level of PSMA, ACADL, NPY, UBE2C, or any combination thereof in a patient or population of patients that does not have metastatic castration resistant prostate cancer.
Embodiment 36. A method of treating metastatic castration resistant prostate cancer comprising:
   to a patient not having an elevated level of PSMA, ACADL, NPY, UBE2C, or any combination thereof, administering a therapeutically effective amount of abiraterone acetate and a therapeutically effective amount of glucocorticoid to treat the metastatic castration resistant prostate cancer.
Embodiment 37. The method of embodiment 36, wherein abiraterone acetate and glucocorticoid are administered concurrently or sequentially.
Embodiment 38. The method of embodiment 36 or 37, wherein the level of PSMA, ACADL, NPY, UBE2C, or any combination thereof is a level of PSMA, ACADL, NPY, or UBE2C mRNA.
Embodiment 39. The method of any one of embodiments 36-38, wherein the patient does not have an elevated level of PSMA, ACADL, NPY, UBE2C, or any combination thereof compared to a control.
Embodiment 40. The method of embodiment 39, wherein the control comprises a previous level of PSMA, ACADL, NPY, UBE2C, or any combination thereof from the patient.
Embodiment 41. The method of embodiment 40, wherein the control comprises a level of PSMA, ACADL, NPY, UBE2C, or any combination thereof in a patient or population of patients that does not have metastatic castration resistant prostate cancer.
Embodiment 42. Use of PSMA, ACADL, NPY, UBE2C, or any combination thereof in predicting sensitivity or acquired resistance to abiraterone acetate-glucocorticoid treatment in a patient having metastatic castration resistant prostate cancer.
Embodiment 43. PSMA, ACADL, NPY, UBE2C, or any combination thereof for use in predicting sensitivity or acquired resistance to abiraterone acetate-glucocorticoid treatment in a patient having metastatic castration resistant prostate cancer.
Embodiment 44. The use of embodiment 42 or 43, wherein the PSMA, ACADL, NPY, UBE2C, or any combination thereof is mRNA obtained from a biological sample from the patient, wherein the patient has received abiraterone acetate and glucocorticoid treatment.
Embodiment 45. The method of any one of embodiments 1-41 or the use of any one of embodiments 42-44, wherein the glucocorticoid is prednisone, prednisolone, hydrocortisone, dexamethasone, cortisone, or methylprednisolone.

What is claimed:

1. A method of diagnosing and treating abiraterone acetate-glucocorticoid sensitive metastatic castration resistant prostate cancer in a patient comprising:
in a patient who has received abiraterone acetate and glucocorticoid treatment, diagnosing the patient as having abiraterone acetate-glucocorticoid sensitive metastatic castration resistant prostate cancer when a level of PSMA, ACADL, NPY, UBE2C, or any combination thereof in a biological sample of the patient is not elevated; and
treating the diagnosed patient with abiraterone acetate and glucocorticoid.

2. The method of claim 1, further comprising, prior to the diagnosing, detecting a level of PSMA, ACADL, NPY, UBE2C, or any combination thereof in a biological sample of the patient.

3. The method of claim 2, wherein the biological sample comprises circulating tumor cells.

4. The method of claim 1, wherein the level of PSMA, ACADL, NPY, or UBE2C is a level of PSMA, ACADL, NPY, or UBE2C mRNA.

5. The method of claim 1, further comprising, prior to the diagnosis, comparing the level of PSMA, ACADL, NPY, UBE2C, or any combination thereof to a control.

6. The method of claim 5, wherein the control comprises a previous level of PSMA, ACADL, NPY, UBE2C, or any combination thereof from the patient.

7. The method of claim 5, wherein the control comprises a level of PSMA, ACADL, NPY, UBE2C, or any combination thereof from a patient or population of patients that does not have metastatic castration resistant prostate cancer.

8. A method of treating metastatic castration resistant prostate cancer in a patient comprising:
administering abiraterone acetate and glucocorticoid to the patient to treat the metastatic castration resistant prostate cancer, wherein the patient does not have an elevated level of ACADL, NPY, UBE2C, or any combination thereof.

9. The method of claim 8, wherein the patient has received abiraterone acetate and glucocorticoid treatment.

10. The method of claim 9, wherein the patient does not have an elevated level of PSMA.

11. The method of claim 8, wherein abiraterone acetate and glucocorticoid are administered concurrently or sequentially.

12. The method of claim 10, wherein the level of PSMA, ACADL, NPY, UBE2C, or any combination thereof is a level of PSMA, ACADL, NPY, or UBE2C mRNA.

13. The method of claim 10, wherein the patient does not have an elevated level of PSMA, ACADL, NPY, UBE2C, or any combination thereof compared to a control.

14. The method of claim 13, wherein the control comprises a previous level of PSMA, ACADL, NPY, UBE2C, or any combination thereof from the patient.

15. The method of claim 13, wherein the control comprises a level of PSMA, ACADL, NPY, UBE2C, or any combination thereof in a patient or population of patients that does not have metastatic castration resistant prostate cancer.

16. A method of treating metastatic castration resistant prostate cancer in a patient comprising:
administering abiraterone acetate and glucocorticoid to the patient to treat the metastatic castration resistant prostate cancer, wherein the patient has received abiraterone acetate and glucocorticoid treatment and wherein the patient does not have an elevated level of PSMA, ACADL, NPY, UBE2C, or any combination thereof.

17. The method of claim 16, wherein the abiraterone acetate and glucocorticoid are administered concurrently or sequentially.

18. The method of claim 16, wherein the level of PSMA, ACADL, NPY, UBE2C, or any combination thereof is a level of PSMA, ACADL, NPY, or UBE2C mRNA.

19. The method of claim 16, wherein the patient does not have an elevated level of PSMA, ACADL, NPY, UBE2C, or any combination thereof compared to a control.

20. The method of claim 19, wherein the control comprises a previous level of PSMA, ACADL, NPY, UBE2C, or any combination thereof from the patient.

21. The method of claim 19, wherein the control comprises a level of PSMA, ACADL, NPY, UBE2C, or any combination thereof in a patient or population of patients that does not have metastatic castration resistant prostate cancer.
